SEQUENCE LISTING (22 sequences)

SEQ ID NO: 1 - protein, 8 aa, Artificial sequence
  VH CDR1
GYTFTSYA

SEQ ID NO: 2 - protein, 8 aa, Artificial sequence
  VH CDR2
INTNTGNP

SEQ ID NO: 3 - protein, 19 aa, Artificial sequence
  VH CDR3
ARPQGGSSWYRDYYYGMDV

SEQ ID NO: 4 - protein, 6 aa, Artificial sequence
  VL CDR1
QSISSW

SEQ ID NO: 5 - protein, 3 aa, Artificial sequence
  VL CDR2
KAS

SEQ ID NO: 6 - protein, 9 aa, Artificial sequence
  VL CDR3
QQYNSYPYT

SEQ ID NO: 7 - protein, 126 aa, Artificial sequence
  VH
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARPQGGSSWYRDYYYGMDVWGQGTTVTVSS

SEQ ID NO: 8 - protein, 107 aa, Artificial sequence
  VL
DIQMTQSPSTLSASVGDRVTITCRASQSISSWLAWYQQKPGKAPKLLIYKASSLESGVPSRFSGSGSGTEFTLTISSLQPDDFATYYCQQYNSYPYTFGQGTKLEIK

SEQ ID NO: 9 - protein, 330 aa, Artificial sequence [sequence omitted]

SEQ ID NO: 10 - protein, 107 aa, Artificial sequence [sequence omitted]

SEQ ID NO: 11 - DNA, 24 bp, Artificial sequence
  VH CDR1 gene
ggctacaccttcacctcctatgct

SEQ ID NO: 12 - DNA, 24 bp, Artificial sequence
  VH CDR2 gene
ataaacaccaacacaggcaaccca

SEQ ID NO: 13 - DNA, 57 bp, Artificial sequence
  VH CDR3 gene
gccagaccacagggaggctcctcctggtacagggactactactatgggatggatgtg

SEQ ID NO: 14 - DNA, 18 bp, Artificial sequence
  VL CDR1 gene
cagagcatctcctcctgg

SEQ ID NO: 15 - DNA, 9 bp, Artificial sequence
  VL CDR2 gene
aaggcatcc

SEQ ID NO: 16 - DNA, 27 bp, Artificial sequence
  VL CDR3 gene
caacaatacaactcctacccatacacc

SEQ ID NO: 17 - DNA, 378 bp, Artificial sequence
  VH gene
caggtccaacttgtccagtctggctctgaactgaaaaagcctggagcctctgtgaaggtgtcctgtaaggcatctggctacaccttcacctcctatgctatgaactgggtgagacaggctcctggacaaggattggagtggatgggctggataaacaccaacacaggcaacccaacctatgcccagggcttcacaggcaggtttgtgttctccctggacacctctgtgagcacagcctacctccaaatctcctccctgaaagcagaggacacagcagtctactactgtgccagaccacagggaggctcctcctggtacagggactactactatgggatggatgtgtggggacaaggcaccacagtgacagtgtcctct

SEQ ID NO: 18 - DNA, 321 bp, Artificial sequence
  VL gene
gacatccagatgacccagagcccaagcaccctgtctgcctctgtgggagacagggtgaccatcacttgtagggcaagccagagcatctcctcctggctggcttggtatcaacagaagcctggcaaggctccaaaactgctgatttacaaggcatcctccttggagtctggagtgccaagcaggttctctggctctggctctggcacagagttcaccctgaccatctcctccctccaacctgatgactttgccacctactactgtcaacaatacaactcctacccatacacctttggacaaggcaccaaattggagattaag

SEQ ID NO: 19 - DNA, 990 bp, Artificial sequence
  CH gene
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccccgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - DNA, 321 bp, Artificial sequence
  CL gene
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 21 - protein, 223 aa, Artificial sequence
  RBD of S protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 22 - protein, 1273 aa, Artificial sequence
  S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2023/0116587 A1
(43) Pub. Date: Apr. 13, 2023

(54) MONOCLONAL ANTIBODY AGAINST NOVEL CORONAVIRUS AND APPLICATION THEREOF

(71) Applicant: PEKING UNIVERSITY, Beijing (CN)

(72) Inventors: Xiaoliang Sunney XIE, Beijing (CN); Yunlong CAO, Beijing (CN); Wenjie SUN, Beijing (CN)

(21) Appl. No.: 17/911,326

(22) PCT Filed: Mar. 12, 2021
(86) PCT No.: PCT/CN2021/080537

§ 371 Date: Sep. 13, 2022

(30) Foreign Application Priority Data
Mar. 13, 2020 (CN) 202010177710.7

(51) Int. Cl. C07K 16/10; A61P 31/14; G01N 33/577; G01N 33/569; C12N 15/63

(52) U.S. Cl. CPC C07K 16/10; A61P 31/14; G01N 33/577; G01N 33/56983; C12N 15/63; A61K 2039/505

(57) ABSTRACT

Provided are a monoclonal antibody against a novel coronavirus and a composition that comprises said antibody. The antibody can be used to diagnose, prevent and/or treat novel coronavirus infections and/or diseases caused by an infection.

### TECHNICAL FIELD

The present invention relates to the field of immunology and molecular virology, and in particular relates to the field of diagnosis, prevention and treatment of a novel coronavirus. Specifically, the present invention relates to a monoclonal antibody against a novel coronavirus, and a composition comprising the antibody (e.g., a diagnostic agent and a therapeutic agent). In addition, the present invention also relates to the use of the antibody. The antibody of the present invention can be used for diagnosing, preventing and/or treating novel coronavirus infections or diseases caused by the infection (e.g., Corona Virus Disease 2019).

### BACKGROUND ART

As a single-stranded RNA virus, novel coronavirus SARS-CoV-2 is the pathogen causing Corona Virus Disease 2019 (COVID-19) and belongs to the family Coronaviridae comprising severe acute respiratory syndrome coronavirus (SARS-CoV) that caused the epidemic situation in 2002-2003 and the Middle East respiratory syndrome coronavirus (MERS-CoV) that caused the epidemic situation in 2012. Coronavirus is a relatively large virus with round, oval or pleomorphic particles having a diameter of 50-200 nm. Coronavirus is an enveloped virus. The capsid of the virus is enveloped with a lipid envelope, on which a wide spike protein (Spike, S protein) is arranged forming a sun halo shape. Studies have confirmed that the S protein is located on the surface of novel coronavirus SARS-CoV-2, and can bind to a receptor, angiotensin converting enzyme 2 (ACE2) molecule of a host cell via a receptor binding domain (RBD) contained therein during the virus infection of the host, thereby initiating fusion of the viral membrane with the host cell membrane and causing the virus to infect the host cell.

So far, a neutralizing antibody has been proved to be an effective method for treating viral diseases. In general, upon stimulated by an antigen, a B lymphocyte in a patient is activated and then transformed and differentiated into a variety of different cells, and antibodies are produced. According to existing researches and reports, there is an antibody against the novel coronavirus in the peripheral blood of patients recovered from Corona Virus Disease 2019, which is produced and secreted by activated B cells. However, there are a variety of B cells in the plasma of the recovered patients, and the binding activities and neutralizing titers of antibodies produced by different B cells are also different. So far, there is no study reporting an antibody against the novel coronavirus with a high binding activity and/or a high neutralizing activity.

Therefore, there is a need to develop an antibody with a high binding activity and/or a high neutralizing activity against novel coronavirus SARS-CoV-2, thereby providing effective means for diagnosing, preventing and/or treating novel coronavirus infections.

### SUMMARY OF THE INVENTION

In the present invention, all scientific and technical terms used herein have the meanings commonly understood by a person skilled in the art unless specified otherwise.

Moreover, laboratory operation steps of cell culture, molecular genetics, nucleic acid chemistry, and immunology used herein are all conventional steps widely used in the corresponding art. To better understand the present invention, definitions and explanations of related terms are provided below.

As used herein, the term “antibody” refers to an immunoglobulin molecule generally consisting of two pairs of polypeptide chains, wherein each pair has one “light” (L) chain and one “heavy” (H) chain. Light chains of an antibody can be classified as a κ light chain and a λ light chain. Heavy chains can be classified as μ, δ, γ, α, and ε, and the isotypes of an antibody are defined as IgM, IgD, IgG, IgA, and IgE, respectively. In light and heavy chains, variable regions and constant regions are connected by a “J” region having about 12 or more amino acids, and a heavy chain also contains a “D” region having about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant region of the antibody can mediate the binding of the immunoglobulin to a host tissue or factor, comprising various cells of the immune system (e.g., effector cells) and the first component of the classical complement system (C1q). VH and VL regions can also be subdivided into regions with high variability (called complementarity determining regions (CDRs)), which are interspersed with more conserved regions called framework regions (FRs). Each VH and VL consists of 3 CDRs and 4 FRs arranged in the following order: FR1, CDR1, FR2, CDR2, FR3, CDR3, and FR4 from amino terminal to carboxy terminal. The variable regions of each heavy/light chain pair (VH and VL) form an antibody binding site, respectively. Distribution of amino acids in various regions or domains follows the definitions in: Kabat Sequences of Proteins of Immunological Interest (National Institutes of Health, Bethesda, Md. (1987 and 1991)), or Chothia & Lesk (1987) J. Mol. Biol. 196:901-917; and Chothia et al. (1989) Nature 342:878-883. The term “antibody” is not limited by any particular method for producing an antibody. For example, the antibody comprises a recombinant antibody, a monoclonal antibody and a polyclonal antibody. The antibody can be antibodies of different isotypes, for example, an IgG (e.g., an IgG1, IgG2, IgG3 or IgG4 subtype), IgA1, IgA2, IgD, IgE or IgM antibody.

As used herein, the term “antigen-binding fragment” of an antibody refers to a polypeptide comprising a fragment of a full-length antibody that retains the ability to specifically bind to the same antigen to which the full-length antibody binds and/or competes with the full-length antibody for specific binding to the antigen, which is also referred to as an “antigen-binding moiety”. See generally, Fundamental Immunology, Ch. 7 Paul, W., ed., 2nd Edition, Raven Press, N.Y. (1989), which is incorporated herein by reference in its entirety for all purposes. An antigen-binding fragment of an antibody can be generated by recombinant DNA techniques or by enzymatic or chemical cleavage of an intact antibody. In some cases, an antigen-binding fragment comprises a Fab, Fab′, F(ab′)₂, Fd, Fv, dAb and complementarity determining region (CDR) fragment, a single chain antibody (e.g., scFv), a chimeric antibody, a diabody and a polypeptide comprising at least a portion of an antibody sufficient to confer a specific antigen-binding ability to the polypeptide.

In some cases, an antigen-binding fragment of an antibody is a single chain antibody (e.g., scFv), wherein VL and VH domains are paired by a connector which enables them to be produced as a single polypeptide chain, thereby forming a monovalent molecule (see, e.g., Bird et al., Science 242:423 426 (1988) and Huston et al., Proc. Natl. Acad. Sci. USA 85:5879 5883 (1988)). Such scFv molecules can have a general structure of NH2-VL-linker-VH-COOH or NH2-VH-linker-VL-COOH. Suitable linkers in the prior art consist of a repeated GGGGS amino acid sequence or a variant thereof. For example, a linker having an amino acid sequence (GGGGS)₄ can be used, and a variant thereof can also be used (Holliger et al. (1993), Proc. Natl. Acad. Sci. USA 90: 6444-6448). Other linkers which can be used in the present invention are described in Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol.

In some cases, an antigen-binding fragment of an antibody is a diabody, i.e., a bivalent antibody, wherein VH and VL domains are expressed on a single polypeptide chain; however, the connector used is too short to allow pairing between the two domains of a same chain, thereby forcing the domain to pair with the complementary domain of another chain and producing two antigen-binding sites (see, e.g., Holliger P. et al., Proc. Natl. Acad. Sci. USA 90:6444 6448 (1993), and Poljak R. J. et al., Structure 2:1121 1123 (1994)).

An antigen-binding fragment of an antibody (e.g., the above-mentioned antibody fragment) can be obtained from a given antibody (e.g., the monoclonal antibody BD23 provided in the present invention) by using conventional techniques known to a person skilled in the art (e.g., recombinant DNA techniques or enzymatic or chemical cleavage) and the antigen-binding fragment of the antibody can be screened for specificity in the same manner as for an intact antibody.

Unless the context clearly dictates, the term “antibody” when referred to herein comprises not only an intact antibody but also an antigen-binding fragment of an antibody.

As used herein, the term “monoclonal antibody” refers to an antibody or a fragment of an antibody from a population of highly homologous antibody molecules, i.e., a population of identical antibody molecules, except for possible naturally occurring mutations. The monoclonal antibody is highly specific for a single epitope on an antigen. Relative to a monoclonal antibody, a polyclonal antibody generally comprises at least 2 or more different antibodies, and these different antibodies generally recognize different epitopes on an antigen. A monoclonal antibody can usually be obtained by using the hybridoma technique first reported by Kohler et al. (Nature, 256:495 ,1975), and can also be obtained by using recombinant DNA techniques (for example, see Journal of virological methods, 2009, 158(1-2): 171-179).

As used herein, a “neutralizing antibody” refers to an antibody or antibody fragment that can clear or significantly reduce virulence (e.g., ability to infect cells) of a target virus.

As used herein, the term “vector” refers to a nucleic acid delivery vehicle into which a polynucleotide can be inserted. When the vector allows for the expression of the protein encoded by the inserted polynucleotide, the vector is called an expression vector. A vector can be introduced into a host cell by transformation, transduction or transfection, and the genetic substance elements carried thereby can be expressed in the host cell. The vector is well known to a person skilled in the art, and comprises but is not limited to: a plasmid; a phagemid; an artificial chromosome such as a yeast artificial chromosome (YAC), a bacterial artificial chromosome (BAC) or a P1-derived artificial chromosome (PAC); a phage such as a λ phage or an M13 phage, and an animal virus. The animal virus that can be used as a vector comprises but is not limited to a retrovirus (comprising a lentivirus), an adenovirus, an adeno-associated virus, a herpes virus (e.g., a herpes simplex virus), a poxvirus, a baculovirus, a papilloma virus and a papovavirus (such as SV40). A vector can contain a variety of elements that control expression, comprising, but not limited to: a promoter sequence, a transcription initiation sequence, an enhancer sequence, a selection element, and a reporter gene. In addition, the vector also can contain a replication initiation site.

As used herein, the term “host cell” refers to a cell that can be used to introduce a vector, comprising but not limited to a prokaryotic cell such as Escherichia coli or Bacillus subtilis, a fungal cell such as a yeast cell or Aspergillus, an insect cell such as Drosophila S2 cell or Sf9, and an animal cell such as a fibroblast, a CHO cell, a COS cell, a NSO cell, an HeLa cell, a BHK cell, an HEK293 cell or a human cell.

As used herein, the term “specifically binding” refers to a non-random binding reaction between two molecules, such as a reaction between an antibody and its corresponding antigen. In certain embodiments, an antibody specifically binding to an antigen (or an antibody specific for an antigen) refers to an antibody that binds to the antigen with an affinity (KD) less than about 10⁻⁵ M, for example less than about 10⁻⁷ M, 10⁻⁸ M, 10⁻⁹M or 10⁻¹⁰ M or less.

As used herein, the term “KD” refers to the dissociation equilibrium constant of a particular antibody-antigen interaction, which is used to describe the binding affinity between an antibody and an antigen. The smaller the equilibrium dissociation constant, the tighter the antibody-antigen binding and the higher the affinity between the antibody and the antigen. Generally, an antibody binds to an antigen with a dissociation equilibrium constant (KD) less than about 10⁻⁵ M. For example, monoclonal antibody BD23 of the present invention can bind to an antigen (e.g., the S protein of a novel coronavirus) with a dissociation equilibrium constant (KD) of about 10⁻⁹M (nM level).

In the present invention, an amino acid is generally represented by one-letter and three-letter abbreviations well known in the art. For example, alanine can be represented by A or Ala.

As used herein, the term “neutralizing activity” refers to the functional activity of an antibody or antibody fragment binding to an antigen protein on a virus, thereby preventing viral infection of cells and/or maturation of viral progeny and/or release of viral progeny. The antibody or antibody fragment with a neutralizing activity can prevent the amplification of the virus, thereby inhibiting or eliminating virus infection.

As used herein, the terms “Corona Virus Disease 2019” and “COVID-19” refer to a pneumonia caused by novel coronavirus infections, both have the same meaning and can be used interchangeably.

After a large number of experimental studies, the inventors of the present application have found an antibody which can specifically recognize and target an S protein of a novel coronavirus, particularly the receptor binding domain (RBD) of the S protein, and shows an efficient ability to neutralize the virus. Therefore, the antibody of the present invention is particularly suitable for diagnosing, preventing and treating novel coronavirus infections or diseases related to the novel coronavirus infections (e.g., Corona Virus Disease 2019).

In a first aspect of the present application, provided is a monoclonal antibody or an antigen-binding fragment thereof, comprising complementarity determining regions 1-3 (CDRs 1-3) of a heavy chain variable region (VH) having amino acid sequences as shown in SEQ ID NOs: 1-3, respectively; and/or, complementarity determining regions 1-3 (CDRs 1-3) of a light chain variable region (VL) having amino acid sequences as shown in SEQ ID NOs: 4-6, respectively.

In some preferred embodiments, the monoclonal antibody comprises a heavy chain variable region (VH) as shown in SEQ ID NO: 7.

In some preferred embodiments, the monoclonal antibody comprises a light chain variable region (VL) as shown in SEQ ID NO: 8.

In some preferred embodiments, the monoclonal antibody comprises: VH CDRs 1-3 having amino acid sequences as shown in SEQ ID NOs: 1-3, respectively, and VL CDRs 1-3 having amino acid sequences as shown in SEQ ID NOs: 4-6, respectively.

In some preferred embodiments, the monoclonal antibody comprises: VH as shown in SEQ ID NO: 7 and VL as shown in SEQ ID NO: 8.

In some preferred embodiments, preferably, the monoclonal antibody or the antigen-binding fragment thereof is selected from a Fab, Fab′, F(ab′)₂, Fd, Fv, dAb, a complementarity determining region fragment, a single chain antibody (e.g., scFv), a human antibody, a chimeric antibody or a bispecific or multispecific antibody.

In some preferred embodiments, the monoclonal antibody further comprises a heavy chain constant region. In some preferred embodiments, the amino acid sequence of the heavy chain constant region is as shown in SEQ ID NO: 9.

In some preferred embodiments, the monoclonal antibody further comprises a light chain constant region. In some preferred embodiments, the amino acid sequence of the light chain constant region is as shown in SEQ ID NO: 10.

In some preferred embodiments, the monoclonal antibody or the antigen-binding fragment thereof can specifically bind to a spike protein (S protein) of a novel coronavirus. In some preferred embodiments, the monoclonal antibody or the antigen-binding fragment thereof can target a receptor binding domain (RBD) of the spike protein (S protein) of the novel coronavirus. In some preferred embodiments, the monoclonal antibody or the antigen-binding fragment thereof can inhibit the receptor binding and/or membrane fusion process mediated by the receptor binding domain (RBD) of the S protein and inhibit the virus infection of a cell.

In some preferred embodiments, the monoclonal antibody or the antigen-binding fragment thereof has a neutralizing ability (for example, capable of neutralizing novel coronavirus). In some preferred embodiments, the monoclonal antibody or the antigen-binding fragment thereof can inhibit novel coronavirus infections or the entry of the novel coronavirus into a host cell. Therefore, the monoclonal antibody or the antigen-binding fragment thereof can neutralize the novel coronavirus, thereby preventing and treating novel coronavirus infections.

The present application also provides an isolated nucleic acid molecule, which encodes the monoclonal antibody or the antigen-binding fragment thereof of the present invention. Such nucleic acid molecules are not limited by the production method therefor, and can be obtained by using genetic engineering recombinant techniques or chemical synthesis methods.

Therefore, in another aspect, the present invention provides an isolated nucleic acid molecule, comprising a nucleotide sequence which can encode a heavy chain variable region of an antibody, wherein the heavy chain variable region of the antibody comprises: VH CDRs 1-3 having amino acid sequences of SEQ ID NOs:1-3, respectively.

In some preferred embodiments, the VH CDRs 1-3 are encoded by nucleotide sequences as shown in SEQ ID NOs:11-13, respectively. Therefore, in some preferred embodiments, the isolated nucleic acid molecule comprises nucleotide sequences as shown in SEQ ID NOs:11-13.

In some preferred embodiments, the heavy chain variable region of the antibody has an amino acid sequence as shown in SEQ ID NO: 7.

In some preferred embodiments, the nucleic acid molecule has a nucleotide sequence as shown in SEQ ID NO: 17.

In another aspect, the present invention provides an isolated nucleic acid molecule, comprising a nucleotide sequence which can encode a light chain variable region of an antibody, wherein the light chain variable region of the antibody comprises: VL CDRs 1-3 having amino acid sequences of SEQ ID NOs: 4-6, respectively.

In some preferred embodiments, the VL CDRs 1-3 are encoded by nucleotide sequences as shown in SEQ ID NOs: 14-16, respectively. Therefore, in some preferred embodiments, the isolated nucleic acid molecule comprises nucleotide sequences as shown in SEQ ID NOs: 14-16.

In some preferred embodiments, the light chain variable region of the antibody has an amino acid sequence as shown in SEQ ID NO: 8.

In some preferred embodiments, the nucleic acid molecule has a nucleotide sequence as shown in SEQ ID NO: 18.

In another aspect, the present invention provides an isolated nucleic acid molecule, comprising the nucleotide sequence which can encode the heavy chain variable region of the antibody as defined above, and the nucleotide sequence which can encode the light chain variable region of the antibody as defined above.

In some preferred embodiments, the heavy chain variable region of the antibody has an amino acid sequence as shown in SEQ ID NO: 7. In some preferred embodiments, the nucleotide sequence which can encode the heavy chain variable region of the antibody has a nucleotide sequence as shown in SEQ ID NO: 17.

In some preferred embodiments, the light chain variable region of the antibody comprises an amino acid sequence as shown in SEQ ID NO: 8. In some preferred embodiments, the nucleotide sequence which can encode the light chain variable region of the antibody has a nucleotide sequence as shown in SEQ ID NO: 18.

In some preferred embodiments, the isolated nucleic acid molecule comprises a nucleotide sequence as shown in SEQ ID NO: 17 and a nucleotide sequence as shown in SEQ ID NO: 18.

In some preferred embodiments, the isolated nucleic acid molecule further comprises a nucleotide sequence which can encode the heavy chain constant region of the antibody. In some preferred embodiments, the heavy chain constant region has an amino acid sequence as shown in SEQ ID NO: 9. In some preferred embodiments, the nucleotide sequence which can encode the heavy chain constant region of the antibody has a nucleotide sequence as shown in SEQ ID NO: 19.

In some preferred embodiments, the isolated nucleic acid molecule further comprises a nucleotide sequence which can encode the light chain constant region of the antibody. In some preferred embodiments, the light chain constant region has an amino acid sequence as shown in SEQ ID NO: 10. In some preferred embodiments, the nucleotide sequence which can encode the light chain constant region of the antibody has a nucleotide sequence as shown in SEQ ID NO: 20.

In another aspect, the present invention provides an isolated nucleic acid molecule encoding the monoclonal antibody or the antigen-binding fragment thereof of the present invention as defined above.

In another aspect, the present invention provides a vector, comprising the isolated nucleic acid molecule as defined above. The vector of the present invention can be a cloning vector and can also be an expression vector. In some preferred embodiments, the vector of the present invention is for example, a plasmid, a cosmid, a phage etc.

In another aspect, also provided is a host cell comprising the isolated nucleic acid molecule or the vector of the present invention. Such host cells comprise, but are not limited to, a prokaryotic cell, for example an Escherichia coli cell, and a eukaryotic cell such as a yeast cell, an insect cell, a plant cell, and an animal cell (such as, a mammal cell, e.g., a mouse cell, a human cell, etc.). The cell of the present invention can also be a cell line, for example, HEK293 cell.

In another aspect, also provided is a method for preparing the monoclonal antibody or the antigen-binding fragment thereof of the present invention, comprising culturing the host cell of the present invention under suitable conditions, and recovering the monoclonal antibody or the antigen-binding fragment thereof of the present invention from a cell culture.

In another aspect, the present invention provides a composition, comprising the monoclonal antibody or the antigen-binding fragment thereof, the isolated nucleic acid molecule, the vector or the host cell as described above.

In another aspect, the present invention provides a kit, comprising the monoclonal antibody or the antigen-binding fragment thereof of the present invention. In some preferred embodiments, the monoclonal antibody or the antigen-binding fragment thereof of the present invention further comprises a detectable label. In some preferred embodiments, the kit further comprises a second antibody, which specifically recognizes the monoclonal antibody or the antigen-binding fragment thereof of the present invention.

Preferably, the second antibody further comprises a detectable label. Such detectable labels are well known to a person skilled in the art and comprise, but are not limited to, a radioisotope, a fluorescent material, a luminescent material, a colored material, an enzyme (e.g., horseradish peroxidase), etc.

In another aspect, the present invention provides a method for detecting presence of a novel coronavirus, an S protein thereof or a RBD of the S protein, or a level thereof in a sample, comprising using the monoclonal antibody or the antigen-binding fragment thereof of the present invention. In some preferred embodiments, the monoclonal antibody or the antigen-binding fragment thereof of the present invention further comprises a detectable label. In another preferred embodiment, the method further comprises detecting the monoclonal antibody or the antigen-binding fragment thereof of the present invention by using a second antibody carrying a detectable label. The method can be used for a diagnostic purpose (for example, the sample is a sample from a patient), or for a non-diagnostic purpose (for example, the sample is a cell sample rather than a sample from a patient).

In another aspect, the present invention provides a method for diagnosing whether a subject is infected with a novel coronavirus, comprising: using the monoclonal antibody or the antigen-binding fragment thereof of the present invention to detect presence of a novel coronavirus, or an S protein thereof or a RBD of the S protein in a sample from the subject. In some preferred embodiments, the monoclonal antibody or the antigen-binding fragment thereof of the present invention further comprises a detectable label. In another preferred embodiment, the method further comprises detecting the monoclonal antibody or the antigen-binding fragment thereof of the present invention by using a second antibody carrying a detectable label.

In another aspect, provided is the use of the monoclonal antibody or the antigen-binding fragment thereof of the present invention in the preparation of a kit, wherein the kit is used for detecting presence of a novel coronavirus, an S protein thereof or a RBD of the S protein, or a level thereof in a sample, or for diagnosing whether a subject is infected with the novel coronavirus.

In some preferred embodiments, the sample comprises, but is not limited to, an excrement, an oral or nasal secretion, an alveolar lavage fluid, etc. from a subject (e.g., mammal, preferably human).

In some preferred embodiments, the monoclonal antibody is an antibody, comprising: VH CDRs 1-3 having amino acid sequences as shown in SEQ ID NOs: 1-3, respectively, and/or VL CDRs 1-3 having amino acid sequences as shown in SEQ ID NOs: 4-6, respectively; and preferably, comprising: VH as shown in SEQ ID NO: 7 and/or VL as shown in SEQ ID NO: 8.

General methods for detecting presence of a target virus or antigen (e.g., a novel coronavirus, or an S protein thereof or a RBD of the S protein) or a level thereof in a sample by using an antibody or an antigen-binding fragment thereof is well known to a person skilled in the art. In some preferred embodiments, the detection method may involve enzyme linked immunosorbent assay (ELISA), enzyme immunodetection, chemiluminescence immunodetection, radioimmunodetection, fluorescence immunodetection, immunochromatography, a competition method, and a similar detection method.

In another aspect, the present invention provides a pharmaceutical composition, comprising the monoclonal antibody or the antigen-binding fragment thereof of the present invention, and a pharmaceutically acceptable carrier and/or excipient. In some preferred embodiments, the monoclonal antibody comprises: VH CDRs 1-3 having amino acid sequences as shown in SEQ ID NOs: 1-3, respectively, and/or VL CDRs 1-3 having amino acid sequences as shown in SEQ ID NOs: 4-6, respectively; and preferably, the monoclonal antibody comprises: VH as shown in SEQ ID NO: 7 and/or VL as shown in SEQ ID NO: 8.

In another aspect, the present invention provides a method for neutralizing virulence of a novel coronavirus in a sample, comprising contacting the sample comprising the novel coronavirus with the monoclonal antibody or the antigen-binding fragment thereof of the present invention. Such methods can be used for therapeutic purposes, or for non-therapeutic purposes (for example, the sample is a cell sample, rather than a sample of or from a patient).

In another aspect, provided is the use of the monoclonal antibody or the antigen-binding fragment thereof of the present invention for preparing a drug, wherein the drug is used for neutralizing virulence of a novel coronavirus in a sample. In another aspect, the present invention provides the monoclonal antibody or the antigen-binding fragment thereof as described above for neutralizing virulence of a novel coronavirus in a sample.

In another aspect, provided is the use of the monoclonal antibody or the antigen-binding fragment thereof of the present invention for preparing a pharmaceutical composition, wherein the pharmaceutical composition is used for preventing or treating novel coronavirus infections or diseases related to the novel coronavirus infections (e.g., Corona Virus Disease 2019) of a subject. In another aspect, the present invention provides the monoclonal antibody or the antigen-binding fragment thereof as described above, for preventing and treating novel coronavirus infections or diseases related to the novel coronavirus infections (e.g., Corona Virus Disease 2019) of a subject.

In another aspect, the present invention provides a method for preventing and treating novel coronavirus infections or diseases related to the novel coronavirus infections (e.g., Corona Virus Disease 2019) of a subject, comprising administering to a subject in need thereof a prophylactically or therapeutically effective amount of the monoclonal antibody or the antigen-binding fragment thereof of the present invention, or the pharmaceutical composition of the present invention.

In some preferred embodiments, the subject is a mammal, for example human.

The monoclonal antibody or the antigen-binding fragment thereof of the present invention, or the pharmaceutical composition of the present invention can be administered to a subject by any suitable routes of administration. Such routes of administration comprise, but are not limited to, oral, buccal, sublingual, topical, parenteral, rectal, intravaginal, or nasal routes.

In some preferred embodiments, the monoclonal antibody is an antibody, comprising: VH CDRs 1-3 having amino acid sequences as shown in SEQ ID NOs: 1-3, respectively, and/or VL CDRs 1-3 having amino acid sequences as shown in SEQ ID NOs: 4-6, respectively; and preferably, comprising: VH as shown in SEQ ID NO: 7 and/or VL as shown in SEQ ID NO: 8.

The drug and pharmaceutical composition provided in the present invention can be used alone or in combination, or can be used in combination with other pharmacologically active agents (e.g., an antiviral drug, such as favipiravir, remdesivir and interferon). In some preferred embodiments, the pharmaceutical composition also contains a pharmaceutically acceptable carrier and/or excipient.

### Sequence Information

The information of partial sequences involved in the present application is as shown in Table 1 below.

TABLE 1
Information of partial sequences

| SEQ | | |
|---|---|---|
| ID | Sequence | |
| NO: | description | Sequence information |
| 1 | VH CDR1 | GYTFTSYA |
| 2 | VH CDR2 | INTNTGNP |
| 3 | VH CDR3 | ARPQGGSSWYRDYYYGMDV |
| 4 | VL CDR1 | QSISSW |
| 5 | VL CDR2 | KAS |
| 6 | VL CDR3 | QQYNSYPYT |
| 7 | VH | QVQLVQSGSELKKPGASVKVSCKASGYTFTSYAMNWVRQAPGQGLEW |
| | | MGWINTNTGNPTYAQGFTGRFVFSLDTSVSTAYLQISSLKAEDTAVYYCA |
| | | RPQGGSSWYRDYYYGMDVWGQGTTVTVSS |
| 8 | VL | DIQMTQSPSTLSASVGDRVTITCRASQSISSWLAWYQQKPGKAPKLLIYK |
| | | ASSLESGVPSRFSGSGSGTEFTLTISSLQPDDFATYYCQQYNSYPYTFGQG |
| | | TKLEIK |
| 9 | CH | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRW |
| | | QQGNVFSCSVMHEALHNHYTQKSLSLSPGK |
| 10 | CL | RTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVT |
| | | KSFNRGEC |
| 11 | VH CDR1 | GGCTACACCTTCACCTCCTATGCT |
| | gene | |
| 12 | VH CDR2 | ATAAACACCAACACAGGCAACCCA |
| | gene | |
| 13 | VH CDR3 | GCCAGACCACAGGGAGGCTCCTCCTGGTACAGGGACTACTACTATGGG |
| | gene | ATGGATGTG |
| 14 | VL CDR1 | CAGAGCATCTCCTCCTGG |
| | gene | |
| 15 | VL CDR2 | AAGGCATCC |
| | gene | |
| 16 | VL CDR3 | CAACAATACAACTCCTACCCATACACC |
| | gene | |
| 17 | VH gene | CAGGTCCAACTTGTCCAGTCTGGCTCTGAACTGAAAAAGCCTGGAGCC |
| | | TCTGTGAAGGTGTCCTGTAAGGCATCTGGCTACACCTTCACCTCCTATGC |
| | | TATGAACTGGGTGAGACAGGCTCCTGGACAAGGATTGGAGTGGATGG |
| | | GCTGGATAAACACCAACACAGGCAACCCAACCTATGCCCAGGGCTTCA |
| | | CAGGCAGGTTTGTGTTCTCCCTGGACACCTCTGTGAGCACAGCCTACCT |
| | | CCAAATCTCCTCCCTGAAAGCAGAGGACACAGCAGTCTACTACTGTGC |
| | | CAGACCACAGGGAGGCTCCTCCTGGTACAGGGACTACTACTATGGGAT |
| | | GGATGTGTGGGGACAAGGCACCACAGTGACAGTGTCCTCT |
| 18 | VL gene | GACATCCAGATGACCCAGAGCCCAAGCACCCTGTCTGCCTCTGTGGGA |
| | | GACAGGGTGACCATCACTTGTAGGGCAAGCCAGAGCATCTCCTCCTGG |
| | | CTGGCTTGGTATCAACAGAAGCCTGGCAAGGCTCCAAAACTGCTGATT |
| | | TACAAGGCATCCTCCTTGGAGTCTGGAGTGCCAAGCAGGTTCTCTGGC |
| | | TCTGGCTCTGGCACAGAGTTCACCCTGACCATCTCCTCCCTCCAACCTG |
| | | ATGACTTTGCCACCTACTACTGTCAACAATACAACTCCTACCCATACACC |
| | | TTTGGACAAGGCACCAAATTGGAGATTAAG |
| 19 | CH gene | GCTAGCACCAAGGGCCCATCGGTCTTCCCCCTGGCACCCTCCTCCAAG |
| | | AGCACCTCTGGGGGCACAGCGGCCCTGGGCTGCCTGGTCAAGGACTA |
| | | CTTCCCCGAACCGGTGACGGTGTCGTGGAACTCAGGCGCCCTGACCAG |
| | | CGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGAC |
| | | CTACATCTGCAACGTGAATCACAAGCCCAGCAACACCAAGGTGGACA |
| | | AGAAAGTTGAGCCCAAATCTTGTGACAAAACTCACACATGCCCACCGT |
| | | GCCCAGCACCTGAACTCCTGGGGGGACCGTCAGTCTTCCTCTTCCCCCC |
| | | AAAACCCAAGGACACCCTCATGATCTCCCGGACCCCTGAGGTCACGTG |
| | | CGTGGTGGTGGACGTGAGCCACGAAGACCCCGAGGTCAAGTTCAACT |
| | | GGTACGTGGACGGCGTGGAGGTGCATAATGCCAAGACAAAGCCGCGG |
| | | GAGGAGCAGTACAACAGCACGTACCGTGTGGTCAGCGTCCTCACCGTC |
| | | CTGCACCAGGACTGGCTGAATGGCAAGGAGTACAAGTGCAAGGTCTC |
| | | CAACAAAGCCCTCCCAGCCCCCATCGAGAAAACCATCTCCAAAGCCAA |
| | | AGGGCAGCCCCGAGAACCACAGGTGTACACCCTGCCCCCATCCCGGG |
| | | ATGAGCTGACCAAGAACCAGGTCAGCCTGACCTGCCTGGTCAAAGGCT |
| | | TCTATCCCAGCGACATCGCCGTGGAGTGGGAGAGCAATGGGCAGCCG |
| | | GAGAACAACTACAAGACCACGCCTCCCGTGCTGGACTCCGACGGCTCC |
| | | TTCTTCCTCTACAGCAAGCTCACCGTGGACAAGAGCAGGTGGCAGCAG |
| | | GGGAACGTCTTCTCATGCTCCGTGATGCATGAGGCTCTGCACAACCACT |
| | | ACACGCAGAAGAGCCTCTCCCTGTCTCCGGGTAAA |
| 20 | CL gene | CGTACGGTGGCTGCACCATCTGTCTTCATCTTCCCGCCATCTGATGAGCA |
| | | GTTGAAATCTGGAACTGCCTCTGTTGTGTGCCTGCTGAATAACTTCTATC |
| | | CCAGAGAGGCCAAAGTACAGTGGAAGGTGGATAACGCCCTCCAATCG |
| | | GGTAACTCCCAGGAGAGTGTCACAGAGCAGGACAGCAAGGACAGCA |
| | | CCTACAGCCTCAGCAGCACCCTGACGCTGAGCAAAGCAGACTACGAG |
| | | AAACACAAAGTCTACGCCTGCGAAGTCACCCATCAGGGCCTGAGCTCG |
| | | CCCGTCACAAAGAGCTTCAACAGGGGAGAGTGT |
| 21 | S protein | RVQPTESIVRFPNITNLCPFGEVFNATRFASVYAWNRKRISNCVADYSVLY |
| | RBD | NSASFSTFKCYGVSPTKLNDLCFTNVYADSFVIRGDEVRQIAPGQTGKIAD |
| | | YNYKLPDDFTGCVIAWNSNNLDSKVGGNYNYLYRLFRKSNLKPFERDIST |
| | | EIYQAGSTPCNGVEGFNCYFPLQSYGFQPTNGVGYQPYRVVVLSFELLHA |
| | | PATVCGPKKSTNLVKNKCVNF |
| 22 | S protein | MFVFLVLLPLVSSQCVNLTTRTQLPPAYTNSFTRGVYYPDKVFRSSVLHST |
| | | QDLFLPFFSNVTWFHAIHVSGTNGTKRFDNPVLPFNDGVYFASTEKSNII |
| | | RGWIFGTTLDSKTQSLLIVNNATNWIKVCEFQFCNDPFLGVYYHKNNKS |
| | | WMESEFRVYSSANNCTFEYVSQPFLMDLEGKQGNFKNLREFVFKNIDGY |
| | | FKIYSKHTPINLVRDLPQGFSALEPLVDLPIGINITRFQTLLALHRSYLTPGD |
| | | SSSGWTAGAAAYYVGYLQPRTFLLKYNENGTITDAVDCALDPLSETKCTL |
| | | KSFTVEKGIYQTSNFRVQPTESIVRFPNITNLCPFGEVFNATRFASVYAWN |
| | | RKRISNCVADYSVLYNSASFSTFKCYGVSPTKLNDLCFTNVYADSFVIRGD |
| | | EVRQIAPGQTGKIADYNYKLPDDFTGCVIAWNSNNLDSKVGGNYNYLY |
| | | RLFRKSNLKPFERDISTEIYQAGSTPCNGVEGFNCYFPLQSYGFQPTNGV |
| | | GYQPYRVVVLSFELLHAPATVCGPKKSTNLVKNKCVNFNFNGLTGTGVLT |
| | | ESNKKFLPFQQFGRDIADTTDAVRDPQTLEILDITPCSFGGVSVITPGTNTS |
| | | NQVAVLYQDVNCTEVPVAIHADQLTPTWRVYSTGSNVFQTRAGCLIGAE |
| | | HVNNSYECDIPIGAGICASYQTQTNSPRRARSVASQSIIAYTMSLGAENS |
| | | VAYSNNSIAlPTNFTISVTTEILPVSMTKTSVDCTMYICGDSTECSNLLLQY |
| | | GSFCTQLNRALTGIAVEQDKNTQEVFAQVKQIYKTPPIKDFGGFNFSQILP |
| | | DPSKPSKRSFIEDLLFNKVTLADAGFIKQYGDCLGDIAARDLICAQKFNGL |
| | | TVLPPLLTDEMIAQYTSALLAGTITSGWTFGAGAALQIPFAMQMAYRFN |
| | | GIGVTQNVLYENQKLIANQFNSAIGKIQDSLSSTASALGKLQDVVNQNA |
| | | QALNTLVKQLSSNFGAISSVLNDILSRLDKVEAEVQIDRLITGRLQSLQTYV |
| | | TQQLIRAAEIRASANLAATKMSECVLGQSKRVDFCGKGYHLMSFPQSAP |
| | | HGVVFLHVTYVPAQEKNFTTAPAICHDGKAHFPREGVFVSNGTHWFVT |
| | | QRNFYEPQIITTDNTFVSGNCDVVIGIVNNTVYDPLQPELDSFKEELDKYF |
| | | KNHTSPDVDLGDISGINASVVNIQKEIDRLNEVAKNLNESLIDLQELGKYE |
| | | QYIKWPWYIWLGFIAGLIAIVMVTIMLCCMTSCCSCLKGCCSCGSCCKFD |
| | | EDDSEPVLKGVKLHYT |

### Beneficial Effects

The monoclonal antibody of the present application (e.g., BD23 antibody) can bind to an S protein of a novel coronavirus with a high affinity, and has a strong neutralizing activity against the novel coronavirus. Therefore, the monoclonal antibody of the present application (e.g., BD23 antibody) has clinical application values for diagnosis, prevention and treatment of novel coronavirus infections.

### BRIEF DESCRIPTION OF THE DRAWINGS

FIG. 1 shows SDS-PAGE detection results of a recombinantly expressed BD23 antibody, wherein “NR” represents non-reducing SDS-PAGE; and “R” represents reducing SDS-PAGE. The results in FIG. 1 show that under non-reducing SDS-PAGE conditions, a single band of about 190.88 KDa was formed; under reducing SDS-PAGE conditions, two bands of about 47.75 KDa and 25.70 KDa (corresponding to heavy and light chains of the antibody, respectively) were formed; in addition, the purified BD23 antibody has a purity of 97.7%.

FIG. 2 shows measurement results of the affinity of BD23 antibody to the S protein detected by using a microscale thermophoresis.

FIG. 3 shows measurement results of the neutralizing inhibitory activity of BD23 antibody against SARS-CoV-2 pseudovirus.

FIG. 4 shows measurement results of the neutralizing inhibitory activity of BD23 antibody against SARS-CoV-2 euvirus.

### DETAILED DESCRIPTION OF EMBODIMENTS

The present invention is described with reference to the following examples, which are meant to illustrate the present invention (but not limit the present invention).

Unless specifically stated, the molecular biology experimental methods and immunodetection methods used in the present invention were basically carried out with reference to J. Sambrook et al., Molecular Cloning: A Laboratory Manual, 2nd Edition, Cold Spring Harbor Laboratory Press, 1989 and F. M. Ausubel et al., Short Protocols in Molecular Biology, 3rd Edition, John Wiley & Sons, Inc., 1995; and restriction enzymes were used according to the conditions recommended by the product manufacturer. If no specific conditions are indicated in the examples, conventional conditions or the conditions suggested by the manufacturer shall be followed. The reagents or instruments used without indicating the manufacturers are commercially available conventional products. It is known to a person skilled in the art that the examples illustrate the present invention by way of example and are not intended to limit the claimed scope of the present invention.

### EXAMPLE 1

### Isolation of Memory B Cell

Blood was collected from people once infected with SARS-CoV-2 virus but recovered and discharged (provided by Beijing Youan Hospital), and PBMCs were extracted using STEMCELL SepMate™-15 (Stemcell Technologies, Cat #86415) in a Biosafety

Physical Containment Level-2+ Laboratory. Then, memory B cells were enriched from the extracted PBMCs using STEMCELL EasySep Human Memory B Cell Isolation Kit (Stemcell Technologies, Cat #17864) according to the manufacturer's instructions.

### EXAMPLE 2

### Acquisition and Identification of Antibody Sequence

Single-cell transcriptome VDJ sequencing of the above-mentioned enriched memory B cells was performed using Chromium Single Cell V(D)J Reagent Kits (purchased from 10× genomics, Cat #100006) according to the manufacturer's instructions. The sequencing results were analyzed, and an antibody was obtained and named as BD23. The sequence information for BD23 antibody is as follows:
the amino acid sequence of the heavy chain variable region is as shown in SEQ ID NO: 7 (the encoding gene thereof is as shown in SEQ ID NO: 17), wherein the amino acid sequences of the CDRs 1-3 of the heavy chain variable region are as shown in SEQ ID NOs: 1-3 (the encoding genes thereof are as shown in SEQ ID NOs: 11-13, respectively);
the amino acid sequence of the light chain variable region is as shown in SEQ ID NO: 8 (the encoding gene thereof is as shown in SEQ ID NO: 18), wherein the amino acid sequences of the CDRs 1-3 of the light chain variable region are as shown in SEQ ID NOs: 4-6 (the encoding genes thereof are as shown in SEQ ID NOs: 14-16, respectively);
the amino acid sequence of the heavy chain constant region is as shown in SEQ ID NO: 9 (the encoding gene thereof is as shown in SEQ ID NO: 19);
and the amino acid sequence of the light chain constant region is as shown in SEQ ID NO: 10 (the encoding gene thereof is as shown in SEQ ID NO: 20).

### EXAMPLE 3

### Preparation and Purification of Antibody BD23

According to the sequence information of BD23 antibody identified in example 2, Sino Biological Inc. was entrusted to express and purify BD23 antibody, and the antigenic reactivity of BD23 antibody was detected.

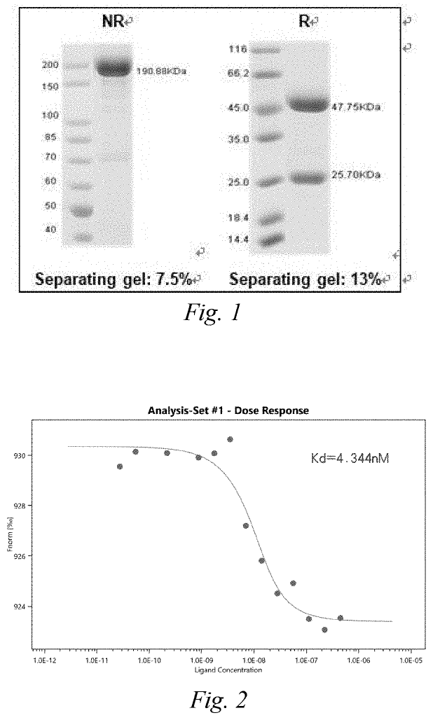

In short, nucleic acid molecules encoding the heavy and light chains of the antibody were synthesized in vitro and then cloned into expression vectors, respectively, thereby obtaining recombinant expression vectors encoding the heavy and light chains of the antibody, respectively. HEK293 cells were co-transfected with the above-mentioned recombinant expression vectors encoding the heavy and light chains of the antibody, respectively. 4-6 hours after the transfection, the cell culture solution was changed to a serum-free medium, which was cultured at 37° C. for another 6 days. After cultivation, the antibody protein expressed by the cells was purified from the culture by an affinity purification column. Then, the purified protein of interest was detected by reducing and non-reducing SDS-PAGE. The results are as shown in FIG. 1. The results in FIG. 1 show that the purified BD23 antibody was obtained with a purity of 97.7%.

Then, the antigenic reactivity of the purified BD23 antibody was detected by ELISA experiments using the RBD of the recombinantly expressed S protein as a coating antigen and using Goat anti-human IgG Fc labeled with horseradish peroxidase (HRP) as a secondary antibody. In short, a 96-well plate was coated with the RBD of the recombinantly expressed S protein (with an amino acid sequence as shown in SEQ ID NO: 21 and a concentration of 0.01 μg/ml or 1 μg/ml), and then the 96-well plate was blocked with a blocking solution. Then, the monoclonal antibodies to be detected (irrelevant control antibody or BD23 antibody; at a concentration of 0.1 μg/ml) were added respectively and incubated. After the plate was washed with an ELISA washing liquid, Goat anti-human IgG Fc labeled with horseradish peroxidase (HRP) was added as a secondary antibody (diluted at 1:500); and the plate was again incubated. Then, the ELISA plate was washed with PBST, and a color developing agent was added to develop the color. Then, the absorbance at OD450 nm was read on a microplate reader. The results are as shown in Table 2. The results in Table 2 show that BD23 antibody can specifically recognize and bind to the RBD of the S protein.

TABLE 2
Reactivity of BD23 antibody with RBD of S
protein detected by ELISA (OD450 reading)

| Sample to | Concentration of RBD protein | |
|---|---|---|
| be detected | 0.01 μg/ml | 1 μg/ml |
| Irrelevant antibody | 0.006 | 0.025 |
| BD23 antibody | 0.017 | 3.026 |

### EXAMPLE 4

### Evaluation of Binding Ability of Antibody BD23 to S Protein

In this example, a high-sensitivity microscale thermophoresis-based molecular interaction analysis system was used to detect the binding ability of antibody BD23 to S protein. The analysis system can be directly used for simple, rapid and precise quantitative analysis of the affinity of biomolecular interactions in a solution.

(1) S Protein with Histidine Tag (His-Tag) Being Labeled with Cy5 Fluorescent Dye

According to the manufacturer's instructions, the recombinantly expressed S protein with His-tag (the amino acid sequence thereof is as shown in SEQ ID NO: 22) was labeled with Cy5 fluorescent dye using a Monolith His-tag labeling kit (Cat#MO-L018). In short, the Cy5 fluorescent dye was diluted to 100 nM using lx PBS-T buffer. Then, 90 μL of S protein with His-tag (at a concentration of 200 nM) was mixed well with 90 μL of the diluted dye (100 nM) and incubated at room temperature for 30 minutes. Then, the incubated sample was centrifuged at 4° C., at 15000 g for 10 minutes. The supernatant was collected into a new tube for use.

### (2) Detection of Binding Affinity of Antibody BD23 to S Protein

According to the manufacturer's instructions, a microscale thermophoresis (MO NT.115PICO) was used to detect the affinity of BD23 antibody to S protein. The specific steps are as follows:
a. antibody BD23 was serially double-diluted (16 concentrations in total), and the initial concentration of dilution was 1 μM. The dilution method is as follows: 16 PCR tubes were prepared, and 10 μl of PBST buffer (PBS+0.005% Tween 20) was added to PCR tubes Nos 2-16; 20 μl of BD23 antibody (at a concentration of 1 μM) was added to tube No 1; 10 μl of the liquid was pipetted from tube No 1, added to tube No 2 and mixed well; and then, 10 μl of the well-mixed liquid was pipetted from tube No 2, added to tube No 3 and mixed well; the operation was performed in sequence, and finally 10 μl of the well-mixed liquid was taken from tube No. 16 and discarded.
b. 10 μl of fluorescent molecules (the S protein labeled with Cy5 fluorescent dye, prepared in step (1)) were added to each PCR tube (tube Nos. 1-16) and mixed well.
c. The mixture was placed at room temperature for 5 minutes, and then the sample was loaded into a microscale thermophoresis using a capillary (cat#MO-K025).
d. The Kd value of the interaction between antibody BD23 and S protein was measured in a microscale thermophoresis using the Binding affinity mode.

The measurement results are as shown in FIG. 2. The results show that the Kd of the interaction between BD23 antibody and S protein was 4.344 nM. This indicates that the BD23 antibody has a very strong affinity with the S protein of the novel coronavirus.

### EXAMPLE 5

### Assessment of Ability of BD23 Antibody to Neutralize SARS-CoV-2 Pseudovirus

In this example, the cell microneutralization assay was used to detect the neutralizing activity of monoclonal antibody BD23 against SARS-CoV-2 pseudovirus with reference to the description of Temperton N J et al., Emerg Infect Dis, 2005, 11(3), 411-416. The SARS-CoV-2 pseudovirus used in this example was provided by China National Institutes for Food and Drug Control, has similar cell infection characteristics to the euvirus, can be used to simulate the early process of euvirus infection of a cell, and carries reporter gene luciferase, which can be quickly and easily detected and analyzed. The safety for operating the pseudovirus is high, and the neutralization experiment can be completed in Biosafety Physical Containment Level-2 Laboratory to detect the neutralization activity (Neutralization titer) of the antibody. The specific steps of the experiment method are as follows:
1. Reagent for Equilibration

The reagent (0.25% trypsin-EDTA, DMEM complete medium) stored at 2° C.-8° C. was taken out and equilibrated at room temperature for more than 30 minutes.
2. Experimental Operation
   (1) A 96-well plate was taken, and the arrangement of the samples was set up as shown in Table 3; A2-H2 wells were set as cell control wells (CC), which only contains experimental cells; A3-H3 wells were set as virus control wells (VV), which contains experimental cells and pseudovirus; A4-A11, B4-B11, C4-C11, D4-D11, E4-E11, F4-F11, G4-G11, H4-H11 wells were set as experimental wells, which contain experimental cells, pseudovirus and different concentrations of antibody to be detected; and other wells were set as blank. The experimental cells and pseudovirus used in this example were Huh-7 cells and SARS-CoV-2 virus (both provided by China National Institutes for Food and Drug Control), respectively.

TABLE 3
Arrangement of samples in 96-well plate

| | 1 | 2 | 3 | 4 | 5-10 | 11 | 12 |
|---|---|---|---|---|---|---|---|
| A | — | CC | VV | Dilution 1 | Dilution 1 | Dilution 1 | — |
| B | — | CC | VV | Dilution 2 | Dilution 2 | Dilution 2 | — |
| C | — | CC | VV | Dilution 3 | Dilution 3 | Dilution 3 | — |
| D | — | CC | VV | Dilution 4 | Dilution 4 | Dilution 4 | — |
| E | — | CC | VV | Dilution 5 | Dilution 5 | Dilution 5 | — |
| F | — | CC | VV | Dilution 6 | Dilution 6 | Dilution 6 | — |
| G | — | CC | VV | Dilution 7 | Dilution 7 | Dilution 7 | — |
| H | — | CC | VV | Dilution 8 | Dilution 8 | Dilution 8 | — |

(2) DMEM complete mediums (containing 1% antibiotic, 25 mM HEPES, 10% FBS) were added at 100 μl/well to the cell control wells; DMEM complete mediums were added at 100 μl/well to the virus control wells; and the indicated concentration of the antibody to be detected diluted in DMEM complete mediums was added to the experimental wells at 50 μ/well. The antibody concentrations of dilutions 1-8 used in Table 3 were 1/30 μg/μl, 1/90 μg/μl, 1/270 μg/μl, 1/810 μg/μl, 1/2430 μg/μl, 1/7290 μg/μl, 1/21870 μg/μl, and 1/65610 μg/μl, respectively.
(3) The SARS-CoV-2 pseudovirus was diluted to about 1.3×10⁴/ml (TCID50) with DMEM complete mediums; and then, the SARS-CoV-2 pseudovirus was added at 50 μl/well to the virus control wells and the experimental wells.
(4) The 96-well plate was placed in a cell incubator (37° C., 5% CO₂) and incubated for 1 hour.
(5) The pre-cultured Huh-7 cells were diluted to 2×10⁵ cells/ml with DMEM complete mediums. After the incubation in the previous step, cells were added at 100 μl/well to the cell control wells, virus control wells and experimental wells.
(6) The 96-well plate was placed in a cell incubator (37° C., 5% CO₂) and cultured for 20-28 hours.
(7) The 96-well plate was taken out from the cell incubator; 150 μl of the supernatant was aspirated from each well and discarded; and then 100 μl of luciferase detection reagents were added, and reacted at room temperature for 2 minutes in the dark.
(8) After the reaction was completed, the liquid in each well was pipetted 6 to 8 times repeatedly using a pipette until the cells were fully lysed. Then, 150 μl of liquid was aspirated from each well and transferred to the corresponding 96-well chemiluminescence detection plate, and the luminescence value was read with a chemiluminescence detector (Perkinelmer EnSight multimode microplate reader).
(9) Calculation of neutralization inhibition rate:

Inhibition rate=[1−(mean luminescence intensity of experimental wells−mean luminescence intensity of CC wells)/(mean luminescence intensity of VV wells−mean luminescence intensity of CC wells)]×100%.
(10) IC50 of the antibody to be detected was calculated by Reed-Muench method according to the result of the neutralization inhibition rate.

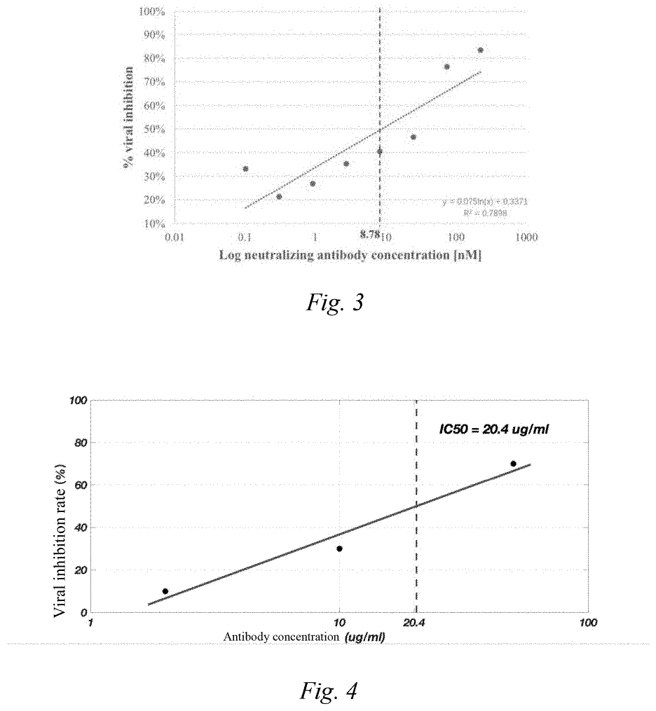

The experimental results are as shown in FIG. 3. The results show that monoclonal antibody BD23 has a good neutralizing activity against SARS-CoV-2 pseudovirus with an IC50 of 8.78 nM (i.e., 1.317 μg/ml).

### EXAMPLE 6

### Assessment of Ability of BD23 Antibody to Neutralize SARS-CoV-2 Euvirus

The SARS-CoV-2 virus used in this example was provided by Academy of Military Medical Sciences, the titer thereof (TCID50) was 10⁵/ml, and all experimental operations were completed in a BSL-3 laboratory. The specific steps of the neutralizing experiment method are as follows:
(1) 100 μl of Vero E6 cells were added to each well of a 96-well culture plate at a concentration of 5×10⁴/ml, and cultured at 37° C., 5% CO2 for 24 hours.
(2) The antibody to be detected was diluted to 3 concentrations: 50 μg/ml, 10 μg/ml and 2 μg/ml. 100 μl of the antibody to be detected at a specified concentration was taken out; an equal volume of SARS-CoV-2 euvirus (100 TCID50) was added; and the mixture was incubated at 37° C., 5% CO₂ for 1 h.
(3) After cultivation in step (1), the cell culture solution in the 96-well culture plate was discarded, and the mixture solution (200 μl) containing the antibody to be detected and the euvirus prepared in step (2) was added as an experimental group. After the mixture was incubated for 1 h, the supernatant was aspirated from the wells, and 200 μl of DMEM mediums (containing 2% antibiotic and 16 μg/ml of trypsin) were added to each well.

During the experiment, the cell control group and the virus control group were set in parallel. In the cell control group (4 replicate wells), after the cell culture solution in the wells was discarded; 200 μl of DMEM mediums (containing 2% antibiotic and 16 μg/ml of trypsin) were added to each well. In the virus control group (4 duplicate wells), after the cell culture solution in the wells was discarded; 100 TCID50 of euvirus (100 μl) was added to each well, and the mixture was incubated at 37° C. for 1 h; After the incubation, the supernatant was aspirated from the wells, and 200 μl of DMEM mediums (containing 2% antibiotic and 16 μg/ml of trypsin) were added to each well.
(4) The cells were cultured for 4-5 days at 37° C., 5% CO₂.
(5) The cytopathic effect (CPE) was observed under the optical microscope, and the inhibitory activities of different concentrations of monoclonal antibody BD23 on CPE were evaluated according to conditions of the cytopathic effect.

The experimental results are as shown in FIG. 4. The results show that monoclonal antibody BD23 has a good neutralizing activity against SARS-CoV-2 euvirus, and can effectively inhibit virus infection and cell invasion (IC50 is 20.4 μg/ml). The inhibition rate of monoclonal antibody BD23 at a concentration of 50 μg/ml against SARS-CoV-2 euvirus was about 70%.

What is claimed is:

1. A monoclonal antibody or an antigen-binding fragment thereof, comprising complementarity determining regions 1-3 (CDRs 1-3) of a heavy chain variable region (VH) having amino acid sequences as shown in SEQ ID NOs: 1-3, respectively; and/or, complementarity determining regions 1-3 (CDRs 1-3) of a light chain variable region (VL) having amino acid sequences as shown in SEQ ID NOs: 4-6, respectively;
preferably, the monoclonal antibody comprises a heavy chain variable region (VH) as shown in SEQ ID NO: 7, and/or a light chain variable region (VL) as shown in SEQ ID NO: 8;
preferably, the monoclonal antibody comprises: VH CDRs 1-3 having amino acid sequences as shown in SEQ ID NOs: 1-3, respectively, and VL CDRs 1-3 having amino acid sequences as shown in SEQ ID NOs: 4-6, respectively;
preferably, the monoclonal antibody comprises: VH as shown in SEQ ID NO: 7 and VL as shown in SEQ ID NO: 8;
preferably, the monoclonal antibody or the antigen-binding fragment thereof is selected from a Fab, Fab′, F(ab′)₂, Fd, Fv, dAb, a complementarity determining region fragment, a single chain antibody (e.g., scFv), a human antibody, a chimeric antibody or a bispecific or multispecific antibody;
preferably, the monoclonal antibody further comprises a heavy chain constant region;
preferably, the amino acid sequence of the heavy chain constant region is as shown in SEQ ID NO: 9;
preferably, the monoclonal antibody further comprises a light chain constant region;
and preferably, the amino acid sequence of the light chain constant region is as shown in SEQ ID NO: 10.

2. An isolated nucleic acid molecule, comprising a nucleic acid sequence that can encode a heavy chain variable region of an antibody, wherein the heavy chain variable region of the antibody comprises: VH CDRs 1-3 having amino acid sequences of SEQ ID NOs:1-3, respectively;
for example, the isolated nucleic acid molecule comprises nucleotide sequences as shown in SEQ ID NOs:11-13;
for example, the heavy chain variable region of the antibody has an amino acid sequence as shown in SEQ ID NO: 7;
and for example, the nucleic acid molecule has a nucleotide sequence as shown in SEQ ID NO: 17.

3. An isolated nucleic acid molecule, comprising a nucleic acid sequence that can encode a light chain variable region of an antibody, wherein the light chain variable region of the antibody comprises: VL CDRs 1-3 having amino acid sequences of SEQ ID NOs: 4-6, respectively;
for example, the isolated nucleic acid molecule comprises nucleotide sequences as shown in SEQ ID NOs: 14-16;
for example, the light chain variable region of the antibody has an amino acid sequence as shown in SEQ ID NO: 8;
and for example, the nucleic acid molecule has a nucleotide sequence as shown in SEQ ID NO: 18.

4. An isolated nucleic acid molecule, which encodes the monoclonal antibody or the antigen-binding fragment thereof of claim 1.

5. A vector, comprising the isolated nucleic acid molecule of any one of claims 2-4.

6. A host cell, comprising the isolated nucleic acid molecule of any one of claims 2-4 or the vector of claim 5.

7. A method for preparing the monoclonal antibody or the antigen-binding fragment thereof of claim 1, comprising culturing the host cell of claim 6 under a suitable condition, and recovering the monoclonal antibody or the antigen-binding fragment thereof from a cell culture.

8. A composition, comprising the monoclonal antibody or the antigen-binding fragment thereof of claim 1, the isolated nucleic acid molecule of any one of claims 2-4, the vector of claim 5, or the host cell of claim 6.

9. A kit, comprising the monoclonal antibody or the antigen-binding fragment thereof of claim 1;
for example, the monoclonal antibody or the antigen-binding fragment thereof further comprises a detectable label, such as a radioisotope, a fluorescent material, a luminescent material, a colored material and an enzyme;
for example, the kit further comprises a second antibody that specifically recognizes the monoclonal antibody or the antigen-binding fragment thereof; and optionally, the second antibody further comprises a detectable label, such as a radioisotope, a fluorescent material, a luminescent material, a colored material and an enzyme.

10. A method for detecting presence of a novel coronavirus, an S protein thereof or a RBD of the S protein, or a level thereof in a sample, comprising using the monoclonal antibody or the antigen-binding fragment thereof of claim 1;
for example, the monoclonal antibody or the antigen-binding fragment thereof further comprises a detectable label, such as a radioisotope, a fluorescent material, a chemiluminescence material, a colored material and an enzyme;
and for example, the method further comprises detecting the monoclonal antibody or the antigen-binding fragment thereof by using a second antibody carrying a detectable label (such as a radioisotope, a fluorescent material, a luminescent material, a colored material and an enzyme).

11. Use of the monoclonal antibody or the antigen-binding fragment thereof of claim 1 in the preparation of a kit, wherein the kit is used for detecting presence of a novel coronavirus, an S protein thereof or a RBD of the S protein, or a level thereof in a sample, or for diagnosing whether a subject is infected with the novel coronavirus;
preferably, the sample is an excrement, an oral or nasal secretion, or an alveolar lavage fluid from the subject (e.g., a mammal, preferably a human).

12. A pharmaceutical composition, comprising the monoclonal antibody or the antigen-binding fragment thereof of claim 1, and a pharmaceutically acceptable carrier and/or excipient;
and preferably, the pharmaceutical composition further comprises a second pharmacologically active agent, such as favipiravir, remdesivir and interferon.

13. A method for neutralizing virulence of a novel coronavirus in a sample, comprising contacting the sample comprising the novel coronavirus with the monoclonal antibody or the antigen-binding fragment thereof of claim 1.

14. Use of the monoclonal antibody or the antigen-binding fragment thereof of claim 1 in the preparation of a drug, wherein the drug is used for neutralizing virulence of a novel coronavirus in a sample or for preventing or treating a novel coronavirus infection or a disease related to the novel coronavirus infection (e.g., Corona Virus Disease 2019) in a subject;
preferably, the subject is a mammal, for example, a human;
and preferably, the drug is used alone or in combination with a second pharmacologically active agent (such as favipiravir, remdesivir and interferon).
